(19) United States
(12) Patent Application Publication
(10) Pub. No.: US 2024/0417711 A1
(43) Pub. Date: Dec. 19, 2024

(54) PAPN MUTANT AND COMPOSITION FOR SITE-DIRECTED MODIFICATION OF PAPN GENE AND APPLICATION THEREOF

(71) Applicant: AGSINO GENSOURCES CO., LTD., Shenzhen (CN)

(72) Inventors: Kui LI, Shenzhen (CN); Yulian MU, Shenzhen (CN); Lei HUANG, Shenzhen (CN)

(21) Appl. No.: 18/490,030

(22) Filed: Oct. 19, 2023

(30) Foreign Application Priority Data
Jun. 14, 2023 (CN) 202310706300.0

(51) Int. Cl. C12N 9/48; A01K 67/027; C12N 9/22; C12N 15/11; C12N 15/90

(52) U.S. Cl. CPC C12N 9/485; A01K 67/0275; C12N 9/22; C12N 15/11; C12N 15/907; C12Y 304/11002; A01K 2217/05; A01K 2227/108; C12N 2310/20

(57) ABSTRACT

The present invention provides a pAPN mutant and a composition for site-directed modification of pAPN gene and application thereof, involving the field of gene editing technology. The mutated pAPN mutant with an alanine at position 734 can maintain its normal expression, while reducing the ability of a host expressing the pAPN mutant to specifically bind to TGEV, which has important scientific and practical significance in pig disease resistance breeding.

### TECHNICAL FIELD

The present invention relates to the field of gene editing technology, in particular to a pAPN mutant and a composition for site-directed modification of pAPN gene and application thereof.

### BACKGROUND

Transmissible gastroenteritis (TGE) is one of the most important diseases that cause morbidity and mortality in piglets, which is a transmissible disease in pigs that must be strictly quarantined as required by the World Organization for Animal Health. TGE is characterized by a short incubation period, rapid transmission speed, and acute onset, so that an infection can be quickly established in a pig herd at various ages from different breeds. Piglets less than 2 weeks old infected with a transmissible gastroenteritis virus (TGEV) have a very high mortality, especially piglets less than 10 days old have a fatality rate up to 100%. Therefore, TGE is considered as one of the important transmissible diseases that harm the pig feeding industry.

TGEV can specifically bind to the host cell, and a S1 moiety of a viral S protein from TGEV as a recognition site can first specifically recognize a receptor on the surface of the host cell, followed by a conformational change in the S protein, so that the virus fuses with the cell membrane. The pAPN protein widely present on the surface of intestinal epithelial cells is an important receptor for TGEV infection in the host cell. Meanwhile, sialic acid as a cofactor plays an important role in the adhesion of TGEV, because sialic acid helps to adhere more virions and promote the virus to cross the mucous layer of intestinal epithelium, so as to protect the virus from emulsification.

However, in addition to playing an important role in mediating TGEV invasion, pAPN also hydrolyzes amide bonds in peptides, amides, and other structures in the small intestine to involve in various peptide metabolisms, thus playing the important role in cell growth, immune regulation, and blood pressure regulation. In addition, the direct knockout of pAPN may affect other physiological functions of the body, as APN is closely related to tumor invasion, migration, immune cell chemotaxis and the like.

A key functional domain or amino acid that mediates the entry of viruses into a host cell by deletion or mutation of a receptor molecule, deprives the receptor of its ability to mediate viruses entry into the cell, while maximizing its function to maintain normal cellular life activities, which is the best strategy to establish TGEV infection resistance targeting a receptor molecule. CRISPR/Cas9 gene editing technology, as a new generation of gene editing technology, can achieve “precise” gene editing, which provides a favorable tool for the construction of a genetically edited pig with TGEV resistance. Therefore, it is particularly important to develop a precisely genetically edited pig with the precise mutation at key sites in pAPN gene, which can maintain normal expression of pAPN protein and resist TGEV infection. This has important scientific and practical significance in pig disease resistance breeding.

In view of this, the present invention is proposed.

### SUMMARY OF THE INVENTION

The first objective of the present invention is to provide a pAPN mutant having an alanine at position 734, and the body expressing the pAPN mutant protein has the ability of TGEV resistance.
The second objective of the present invention is to provide a polynucleotide encoding the above pAPN mutant.
The third objective of the present invention is to provide a composition for site-directed modification of pAPN gene.
The fourth objective of the present invention is to provide use of the composition for site-directed modification of pAPN gene.
The fifth objective of the present invention is to provide a preparation method of a cell with site-directed modification of pAPN gene for non-disease diagnostic and therapeutic purposes.
The sixth objective of the present invention is to provide a pAPN mutated cell.
The seventh objective of the present invention is to provide a preparation method of a genetically edited pig for non-diagnostic and therapeutic purposes.

To solve the above technical problems, the present invention especially adopts the following technical solution:

According to one aspect of the present invention, it provides a pAPN mutant having an alanine at position 734.

Preferably, the pAPN mutant is obtained by mutating pAPN with an amino acid sequence set forth in SEQ ID NO: 1, alternatively, comprising an amino acid sequence that is at least 80% identical with SEQ ID NO: 1;

preferably, the pAPN mutant is a polypeptide having a mutation from threonine to alanine at position 734 in the amino acid sequence set forth in SEQ ID NO: 1.

According to another aspect of the present invention, it further provides a polynucleotide encoding the above pAPN mutant.

Preferably, the polynucleotide encoding the mutation at position 734 in pAPN has a sequence of GCC.

According to another aspect of the present invention, it further provides a composition for site-directed modification of pAPN gene, comprising a first sgRNA, a second sgRNA and a donor DNA; and the first and second sgRNAs each target two respective target sites in pAPN gene;

and the donor DNA mutating T734 to A734 in pAPN contains a site-directed modified fragment of an amino acid at position 734 in pAPN gene, which is located between the two respective target sites;

preferably, the nucleotide sequence encoding a gene at position 734 in pAPN gene has a sequence of GCC, which is achieved by the donor DNA;

preferably, the nucleotide sequence of the donor DNA is set forth in SEQ ID NO: 4;

preferably, the nucleotide sequence encoding the first sgRNA is set forth in SEQ ID NO: 2;

preferably, the nucleotide sequence encoding the second sgRNA is set forth in SEQ ID NO: 3.

preferably, the composition comprises a first vector comprising an expression cassette for expressing the first sgRNA and a second vector comprising an expression cassette for expressing the second sgRNA;
preferably, the first vector further comprises an expression cassette for a genetically edited protein;
preferably, the second vector further comprises an expression cassette for a genetically edited protein;

preferably, the genetically edited proteins expressed by the first and second vector independently comprise Cas9, Cas9n, Cpf1, or C2c2, respectively, and further independently preferably Cas9;

preferably, the backbones of the first and second vector are independently derived from pX330, pX260, pX334, pX335, pX458, pX459, pX461, pX462, pX551, or pX552, respectively; and further independently preferably pX458.

According to another aspect of the present invention, it further provides use of the composition for site-directed modification of pAPN gene in any of the following (a) to (e);
(a) constructing a cell line with site-directed modification of pAPN gene for non-disease diagnostic and therapeutic purposes;
(b) preparing a product for prevention of transmissible gastroenteritis in pigs; and
(c) constructing a pig model with transmissible gastroenteritis resistance for non-disease diagnostic and therapeutic purposes;
(d) a preparation method of a cell with site-directed modification of pAPN gene; and
(e) a preparation method of a genetically edited pig.

preferably, the preparation method of a cell with site-directed modification of pAPN gene comprises that introducing the above composition into a cell of interest to obtain the cell with site-directed modification of pAPN gene;

preferably, the cell of interest is a porcine fibroblast or a porcine ileal epithelial cell;

preferably, a method for introducing comprises electroporation or liposome transfection; further preferably electroporation.

preferably, the cell with site-directed modification of pAPN gene is obtained by screening and identification after the introduction operation;

preferably, the screening comprises screening a monoclonal cell by flow cytometric sorting;

preferably, the identification comprises sequencing or PCR identification;

preferably, PCR identification is performed using primers set forth in SEQ ID NOs: 14-15.

preferably, the pAPN mutated cell expresses the above pAPN mutant; alternatively, it contains the above polynucleotide; alternatively, it is prepared by the above preparation method.

preferably, the preparation method of a genetically edited pig comprises that transplanting the above cell into an enucleated oocyte to obtain a recombinant cloned embryo, which is transplanted into a maternal body for pregnancy to obtain a genetically edited pig with modification of an amino acid at position 734 in pAPN gene;

alternatively, the above composition is microinjected into a zygotic embryo in a pig by microinjection to obtain a pAPN gene-modified embryo, which is transplanted into a maternal body for pregnancy to obtain a genetically edited pig with modification of an amino acid at position 734 in pAPN gene;

preferably, a step of identification after birth is further comprised for the genetically edited pig;

preferably, the identification comprises sequencing or PCR identification;

preferably, PCR identification is performed using primers set forth in SEQ ID NOs: 14-15.

preferably, the above composition for site-directed modification of pAPN gene is used for any one of (a) to (e).

The present invention has the following beneficial effects compared with the prior art:

the pAPN mutant provided by the present invention with an alanine at position 734 can maintain its normal expression, while reducing the ability of a host expressing the pAPN mutant to specifically bind to TGEV.

The composition for site-directed modification of pAPN gene provided by the present invention comprises a first sgRNA, a second sgRNA and a donor DNA, which can effectively cleave two target sites in pAPN gene, replacing the amino acid at position 734 located between the two target sites with a site-directed modified fragment of donor DNA, thereby achieving precise mutation of the amino acid at position 734 in pAPN. Based on the precise modification of pAPN gene while capable of avoiding disruption or alteration of the normal expression of other amino acids in pAPN gene, the present invention maximally retains the physiological activity function of pAPN protein on the basis of resisting TGEV infection, and has advantages of wide applicability and high efficiency for gene editing and the like.

The preparation method of a cell with site-directed modification of pAPN gene using the above composition has advantages of simple operation and low cost with accurate modification of the amino acid at position 734 in pAPN gene in the cell. The preparation method of a genetically edited pig obtained by using the pAPN mutated cell has advantages of convenient operation and wide universality, and the prepared pig with gene editing of the amino acid at position 734 have good TGEV resistance while retaining normal expression of pAPN protein.

### BRIEF DESCRIPTION OF DRAWINGS

The drawings required in the detailed description or the prior art will be briefly introduced below in order to more clearly illustrate the detailed description of the present invention or the technical solution in the prior art. It is obvious that the drawings described below are some embodiments of the present invention, and other drawings can also be obtained based on these drawings without any creative effort for ordinary persons in the art.

FIG. 1 is an expression graph showing overexpressed pAPN proteins in porcine ileal epithelial cells with precise modification of amino acids at positions 729 and 734 in pAPN gene, as provided in Example 1 of the present invention;

FIG. 2 is an expression graph showing overexpressed pAPN proteins in porcine ileal epithelial cells with precise modification of an amino acid at position 735 in pAPN gene, as provided in Example 1 of the present invention;

FIG. 3 is a graph of the results of fluorescence quantitative PCR (qPCR) for detection of the copy number of TGEV RNA in overexpressed porcine ileal epithelial cells infected with TGEV, in which amino acids at positions 729, 734 and 735 in pAPN gene are precisely modified, as provided in Example 1 of the present invention;

FIG. 4 is a graph of the results of Western Blot for detection of TGEV-N protein in overexpressed porcine ileal epithelial cells infected with TGEV, in which amino acids at positions 729 and 734 in pAPN gene are precisely modified, as provided in Example 1 of the present invention;

FIG. 5 is a graph of the results of Western Blot for detection of TGEV-N protein in overexpressed porcine ileal epithelial cells infected with TGEV, in which amino acids at position 735 in pAPN gene are precisely modified, as provided in Example 1 of the present invention;

FIG. 6 is a graph of the results of indirect immunofluorescence assay (IFA) for detection of pAPN and TGEV expressions in overexpressed porcine ileal epithelial cells infected with TGEV, in which amino acids at positions 729, 734 and 735 in pAPN gene are precisely modified, as provided in Example 1 of the present invention;

FIG. 7 is a graph of the results of TCID50detection in overexpressed porcine ileal epithelial cells infected with TGEV, in which amino acids at positions 729, 734 and 735 in pAPN gene are precisely modified, as provided in Example 1 of the present invention;

FIG. 8 is a pattern diagram of the precise mutation of an amino acid at position 734 in pig pAPN gene, as provided in Example 2 of the present invention;

FIG. 9 is a graph of the results of sequencing for a porcine ileal epithelial cell monoclonal with precise modification of an amino acid at position 734 in pAPN gene, as provided in Example 3 of the present invention;

FIG. 10 is a graph of the results of qPCR for detection of the resistance of a porcine ileal epithelial cell with precise modification of an amino acid at position 734 in pAPN gene to TGEV infection, as provided in Example 3 of the present invention;

FIG. 11 is a graph of the results of IFA for detection of the resistance of a porcine ileal epithelial cell with precise modification of an amino acid at position 734 in pAPN gene to TGEV infection, as provided in Example 3 of the present invention;

FIG. 12 is a graph of the results of sequencing for a porcine fibroblast with precise modification of an amino acid at position 734 in pAPN gene, as provided in Example 4 of the present invention;

FIG. 13 is a graph of the results of sequencing for a genetically edited pig with precise modification of an amino acid at position 734 in pAPN gene, as provided in Example 5 of the present invention;

### DETAILED DESCRIPTION

The technical solution of the present invention will be described clearly and completely by combining with examples below, and it is obvious that the described examples are part of the examples in the present invention, but not all of them. Based on the examples in the present invention, all other examples obtained by ordinary technicians in the art without creative efforts fall within the scope of protection of the present invention.

According to one aspect of the present invention, it provides a pAPN mutant having an alanine at position 734. The mutant provided by the present invention is obtained by mutating at position 734 in pAPN with an alanine. The present invention does not limit whether the pAPN without mutation contains other mutation sites, so the precursor of the pAPN mutant provided by the present invention can be a wild-type pAPN or a pAPN mutant that has mutated at other sites based on the wild-type pAPN. The precursor of the pAPN mutant is considered a pAPN protein according to the general definition in the art.

In an optional embodiment, the amino acid sequence of pAPN mutant is set forth in SEQ ID NO: 1, alternatively, comprises an amino acid sequence that is at least 80% identical with SEQ ID NO: 1, for example, but not limited to an amino acid sequence that is at least 80%, 85%, 90%, 95%, or 98% identical with SEQ ID NO: 1.

In an optional embodiment, the pAPN mutant has a mutation from threonine to alanine at position 734 in the amino acid sequence set forth in SEQ ID NO: 1.

According to another aspect of the present invention, it further provides a polynucleotide encoding the above pAPN mutant. “Polynucleotide” herein refers to a polymeric form of a nucleotide including a ribonucleotide and/or a deoxyribonucleotide with any length. Examples of the polynucleotide include, but are not limited to, single-stranded, double-stranded, or multi-stranded DNA or RNA, genomic DNA, cDNA, DNA-RNA heterozygote, or a polymer comprising purine and pyrimidine bases or other natural, chemical, or biochemical modified, unnatural, or derived nucleotide bases. The polynucleotide encodes the above pAPN mutant, which optionally encode a sense or antisense strand. The polynucleotide can be naturally occurring, synthesized, recombinant, or any combination thereof. In an optional embodiment, the polynucleotide encoding the mutation at position 734 in pAPN where an alanine is expressed has a sequence of GCC.

According to another aspect of the present invention, it further provides a composition for site-directed modification of pAPN gene.

The composition comprises a first sgRNA, a second sgRNA and a donor DNA, and the first and second sgRNAs each target two respective target sites in pAPN gene, which can allow the genetically edited protein to target the sequence near the amino acid at position 734 in pAPN gene, but its specific sequence is not defined, as long as allowing for precise targeting function.

In an optional embodiment, the nucleotide sequence encoding the first sgRNA is set forth in SEQ ID NO: 2. In an optional embodiment, the nucleotide sequence encoding the second sgRNA is set forth in SEQ ID NO: 3. The sgRNAs having sequences in SEQ ID NOs: 2 and 3 have more potent targeting and more precise modification.

The composition further comprises a donor DNA containing a site-directed modified fragment of an amino acid at position 734 in pAPN gene that is used to replace the amino acid at position 734 in pAPN gene in order to mutate the amino acid at position 734 in pAPN to A734, thereby achieving sequence recombination, and the site-directed modified fragment is located between two targets targeted by the first and second sgRNAs. The specific sequence of the donor DNA is not defined, as long as allowing for an amino acid mutation at position 734.

In an optional embodiment, the nucleotide sequence encoding a gene at position 734 in pAPN gene has a sequence of GCC, which is achieved by the donor DNA, further preferably, the ACT encoding the amino acid at position 734 in the polynucleotide of pAPN is replaced with GCC by the donor DNA. In an optional embodiment, the nucleotide sequence of the donor DNA capable of accurate replacement of the amino acid at position 734 encoded by pAPN gene with A734 (alanine) is set forth in SEQ ID NO: 4.

In an optional embodiment, the nucleotide sequence encoding the first sgRNA is set forth in SEQ ID NO: 2, the nucleotide sequence encoding the second sgRNA is set forth in SEQ ID NO: 3, and the nucleotide sequence of the donor DNA is set forth in SEQ ID NO: 4 in the above composition.

In the composition for site-directed modification of pAPN gene provided by the present invention, both the first and second sgRNAs can target a fragment of interest that is enzymatically cleaved by the genetically edited protein. Sequence recombination is realized by using donor DNA again. The donor DNA is a replacement template for the modified target sequence of interest, which can specifically recognize the sequence near the amino acid site at position 734 in pAPN gene under the guidance of the first and second sgRNAs. Based on the genetically edited protein, the target fragment is enzymatically cleaved and the donor DNA sequence is guided to replace an original homologous fragment in the cell, thereby achieving the purpose of precise modification of the amino acid at position 734 in pAPN gene. Based on the precise modification of the amino acid at position 734 in pAPN gene while capable of avoiding disruption or alteration of the normal expression of other amino acids in pAPN gene, the present invention maximally retains the physiological activity function of pAPN protein on the basis of resisting TGEV infection, and has advantages of wide applicability and high efficiency for gene editing and the like, which provides strong support for the preparation and breeding of new TGEV-resistant pig varieties with a single amino acid precise mutation in pAPN.

It should be noted that the composition for site-directed modification of pAPN gene provided by the present invention can be used in any form acceptable in the art in combination with a genetically edited protein or a polynucleotides expressing the genetically edited protein. Based on the genetically edited protein, effectively enzymatic cleavage in various cells can be performed and the recombination of the cleaved sequence is guided, which has advantages of wide applicability and high efficiency for enzymatic cleavage. Types of genetically edited proteins are not defined by the present invention, as long as allowing for the genome editing function.

In an optional embodiment, the composition comprises a first vector comprising an expression cassette for expressing the first sgRNA and a second vector comprising an expression cassette for expressing the second sgRNA. In an optional embodiment, the first vector further comprises an expression cassette for a genetically edited protein, and the genetically edited protein expressed by the first vector includes, but are not limited to, Cas9, Cas9n, Cpf1, or C2c2, preferably Cas9. In an optional embodiment, the backbone of the first vector is derived from, for example, but not limited to pX330, pX260, pX334, pX335, pX458, pX459, pX461, pX462, pX551, or pX552, preferably pX458.

In an optional embodiment, the second vector further comprises an expression cassette for a genetically edited protein, and the genetically edited protein expressed by the first vector includes, but are not limited to, Cas9, Cas9n, Cpf1, or C2c2, preferably Cas9. In an optional embodiment, the backbone of the second vector is derived from, for example, but not limited to pX330, pX260, pX334, pX335, pX458, pX459, pX461, pX462, pX551, or pX552, preferably pX458.

Cas9 and pX458 have characteristics of wide universality, good versatility, and high product maturity, thus higher efficiency for enzymatic cleavage can be achieved by using pX458 as the backbone of the vector for gene editing.

In an optional embodiment, the single strands of oligonucleotides with sequences set forth in SEQ ID NOs: 9-10 and SEQ ID NOs: 11-12 is annealed, respectively, in order to form double strands, each of which is linked to the backbone of the enzymatically cleaved vector, and the first and second vectors are obtained by screening for positive clones.

According to another aspect of the present invention, it further provides use of the above composition in any of the following (a) to (e);
(a) constructing a cell line with site-directed modification of pAPN gene for non-disease diagnostic and therapeutic purposes;
(b) preparing a product for prevention of transmissible gastroenteritis in pigs; and
(c) constructing a pig model with transmissible gastroenteritis resistance for non-disease diagnostic and therapeutic purposes;
(d) a preparation method of a cell with site-directed modification of pAPN gene; and
(e) a preparation method of a genetically edited pig.

The site-directed modification of pAPN gene can be achieved by the composition for site-directed modification of pAPN gene provided by the present invention. This system can be used to construct a cell line with site-directed modification of pAPN gene. Since T734 is the most important amino acid site that affects the activity of a TGEV receptor, its point mutation can block the binding of pAPN and TGEV, so as to resist the infection of TGEV, thereby greatly enhancing the body's resistance to TGEV, and constructing a pig with transmissible gastroenteritis resistance. For ease of use, it is prepared into product forms such as a kit and the like.

In an optional embodiment, the preparation method of a cell with site-directed modification of pAPN gene comprises that introducing the above composition for site-directed modification of pAPN gene into a cell of interest to obtain the cell with site-directed modification of pAPN gene. Among them, a method for introducing includes, but are not limited to, electroporation or liposome transfection, preferably electroporation with higher transfection efficiency. The cell of interest includes, but are not limited to, a porcine fibroblast, preferably a porcine fetal fibroblast due to its higher cloning efficiency compared to other cells, or a porcine ileal epithelial cell.

In an optional embodiment, the preparation method further includes obtaining the cell with site-directed modification of pAPN gene by screening and identification after the introduction operation. Preferably, the monoclonal cell is screened by flow cytometric sorting and identified whether it is the cell with site-directed modification at position 734 in pAPN gene, preferably by sequencing.

In an optional embodiment, DNA from the monoclonal cell can be extracted, followed by PCR amplification using primers set forth in SEQ ID NOs: 14-15 to obtain the amplified products, which can be sequenced to confirm whether the cell with precise modification.

In an optional embodiment, the pAPN mutated cell comprises a cell capable of expressing the pAPN mutant in the above embodiment; alternatively, the cell contains the polynucleotide encoding the pAPN mutant in the above embodiment, which can be expressed or not expressed in the pAPN mutated cell, and it can only be replicated but not expressed in the pAPN mutated cell. In an optional embodiment, the pAPN mutated cell is prepared using the preparation method of the above cell with site-directed modification of pAPN gene for non-disease diagnostic and therapeutic purposes.

In an optional embodiment, the preparation method includes transplanting the above pAPN mutated cell into an enucleated oocyte to obtain a recombinant cloned embryo, which is transplanted into a maternal body for pregnancy to obtain a genetically edited pig with modification of an amino acid at position 734 in pAPN gene.

In other optional embodiments, the above composition for site-directed modification of pAPN gene is microinjected into a zygotic embryo in a pig by microinjection to obtain a pAPN gene-modified embryo, which is transplanted into a maternal body for pregnancy to obtain a genetically edited pig with modification of an amino acid at position 734 in pAPN gene

In an optional embodiment, a step of identification after birth is further included for the genetically edited pig, preferably, sequencing is used to identify whether the genetically edited pig express the pAPN mutant.

In an optional embodiment, DNA from the genetically edited pig can be extracted, followed by PCR amplification using primers set forth in SEQ ID NOs: 14-15 to obtain the amplified products, which can be sequenced to confirm whether the pig with precise modification.

In an optional embodiment, the above composition for site-directed modification of pAPN gene is used for any one of (a) to (e).

The present invention is further illustrated by specific examples below, but it should be understood that these examples are intended only for more detailed illustration purposes and should not be understood to limit the present invention in any form.

### Main Reagents

Collagenase type IV for isolation of porcine fetal fibroblasts were purchased from sigma; DMEM, FBS, PS, NEAA, and Glutamine for cell culture were all purchased from Gibco; the DNA kit for extracting cells and ear tissues was purchased from Tiangen Biotech Co., Ltd.; primers were synthesized by Beijing Tsingke Biotech Co., Ltd.; and the KOD FX PCR enzyme for PCR was purchased from TOYOBO.

Porcine ileal epithelial cells with pAPN gene knockout (Immortal Pig Intestinal-2I Knock Out, IPI-2I-KO) could be found in the reference of “Xu Changjiang, Wang Xiaopeng, Xu Kui et al. Establishment of pAPN gene knockout IPI-2I cell lines Mediated by CRISPR/Cas9 System [J]. China Animal Husbandry and Veterinary Medicine, 2021,48(7):2282-2290.DOI:10.16431/j.cnki.1671-7236.2021.07.002.”.

### Main Instruments

CO₂ incubator (Thermo Scientific, 3111); clean bench (AIRTECH, SW-CJ-1FD); inverted fluorescence microscope (ZEISS, observerA1); PCR instrument (BIO-RID, C1000 Touch); gel imaging system (BIO-RID, Universal Hood II); micromanipulation system (Eppendorf, Celltram vario); flow cytometric sorter (BD, Aria III).

### Example 1 Establishment and Functional Verification of Overexpressed Porcine Ileal Epithelial Cells with Precise Modification of Amino Acids at Positions 729, 734 and 735 in pAPN Gene

I. Establishment of overexpressed cells with precise modification of amino acids at positions 729, 734 and 735 in pAPN gene and detection of pAPN expressions in them:
1. CDS sequences of wild-type pAPN gene (SEQ ID NO: 5), as well as pAPN genes with precise modification of amino acids at positions 729, 734 and 735 (SEQ ID NO: 6, SEQ ID NO: 7, SEQ ID NO: 8) named PLVX-WT, PLVX-729, PLVX-734, and PLVX-735, respectively, were separately linked to the backbones of PLVX vectors, followed by plasmid sequencing for future use after expansion.
2. On the day before electrotransfection, porcine ileal epithelial cells with pAPN gene knockout (Immortal Pig Intestinal-2I Knock Out, IPI-2I-KO) were recovered into 10 cm dishes, and the cell transfection could be performed until to about 80% confluence of cells.
3. PLVX-WT, PLVX-729, PLVX-734, PLVX-735, and PLVX empty vector were electrotransfected into IPI-2I-KO cells to obtain successfully overexpressed cells with precise modification, named IPI-2I-WTOE, IPI-2I -729OE, IPI-2I-734OE, IPI-2I-735OE, and IPI-2I-Vector, respectively, for use as donor cells in subsequent TGEV infection experiments.

### II. Functional Verification

IPI-2I-WTOE, IPI-2I-729OE, IPI-2I-734OE, IPI-2I-735OE, and IPI-2I-Vector cells obtained in step 1 above were transfected for 24 h, followed by TGEV infection testing with the specific steps as follows:
1. IPI-2I-WTOE, IPI-2I-729OE, IPI-2I-734OE, IPI-2I-735OE, and IPI-2I-Vector cells were inoculated with TGEV virus strains (MOI=1), respectively. Meanwhile, IPI-2I-KO cells without virus inoculation were used as the Mock group.
2. Cells were collected at 12 h after infection, followed by extraction of cell proteins for detection of the expression of pAPN by Western blot. Meanwhile, IPI-2I-KO cells without virus inoculation were used as the Mock group.

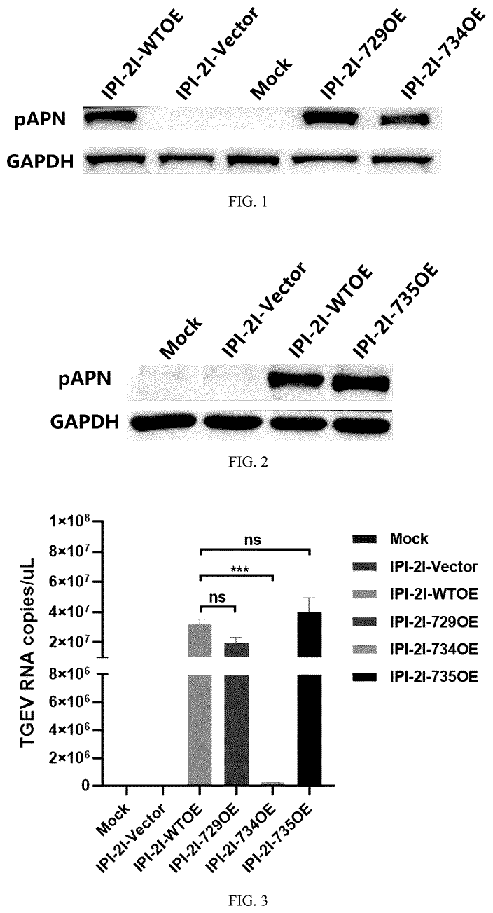

The results of pAPN protein detection were shown in FIGS. 1 and 2, indicating that pAPNs were normally expressed in the IPI-2I-WTOE, IPI-2I-729OE, IPI-2I-734OE, and IPI-2I-735OE groups.
3. Cells were collected at 12 h after infection, and washed 4-5 times with PBS, followed by extraction of RNA for detection of the copy number of TGEV virus in cells by qPCR. Meanwhile, IPI-2I-KO cells without virus inoculation were used as the Mock group.

The qRT-PCR results were shown in FIG. 3, indicating that there were no significant changes in the copy number of TGEV genomic RNA in IPI-2I-729OE and IPI-2I-735OE cells (P>0.05), while the copy number of TGEV genomic RNA in IPI-2I-734OE cells was significantly reduced (***P<0.001), compared with IPI-2I-WTOE cells.
4. Cells were collected at 12 h after infection, followed by extraction of cell proteins for detection of the expression of TGEV-N protein by Western blot. Meanwhile, IPI-2I-KO cells without virus inoculation were used as the Mock group.

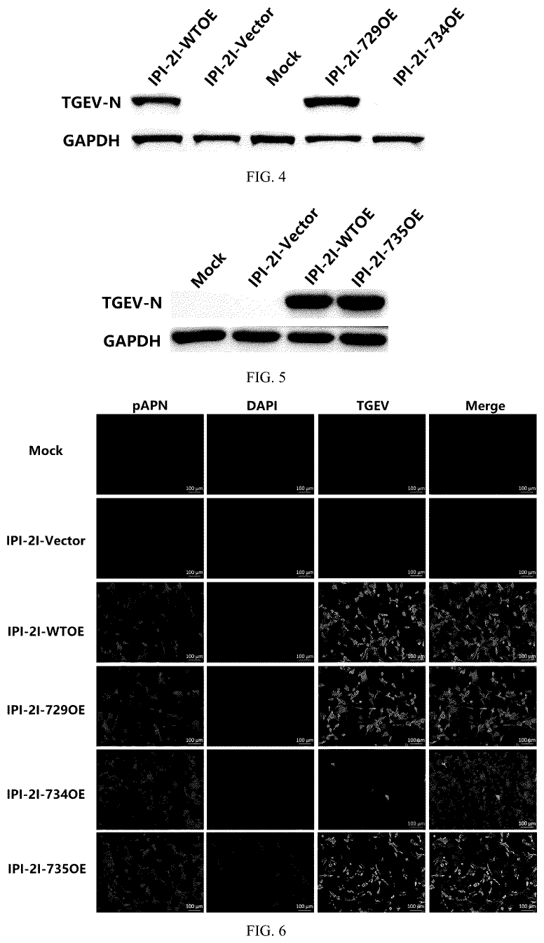

The expression of TGEV-N protein was shown in FIGS. 4 and 5, indicating that there was no significant change in the expression of TGEV-N protein in IPI-2I-729OE and IPI-2I-735OE cells, while the expression of TGEV-N protein in IPI-2I-734OE cells was significantly reduced, compared with IPI-2I-WTOE cells.
5. Cells were collected at 12 h after infection, followed by detection of TGEV infection in cells by indirect immunofluorescence assay (IFA). Meanwhile, IPI-2I-KO cells without virus inoculation were used as the Mock group.

The IFA detection results were shown in FIG. 6, indicating that IPI-2I-WT cells were infected with a large number of TGEVs after virus inoculation; and there was no significant change in the amount of TGEV infection in the IPI-2I-729OE and IPI-2I-735OE groups, while the amount of TGEV infection in IPI-2I-734OE cells was significantly reduced compared with IPI-2I-WT cells.
6. Cells were collected at 12 h after infection, followed by detection of the TGEV virus titer in cells by the TCID50 method. Meanwhile, IPI-2I-KO cells without virus inoculation were used as the Mock group.

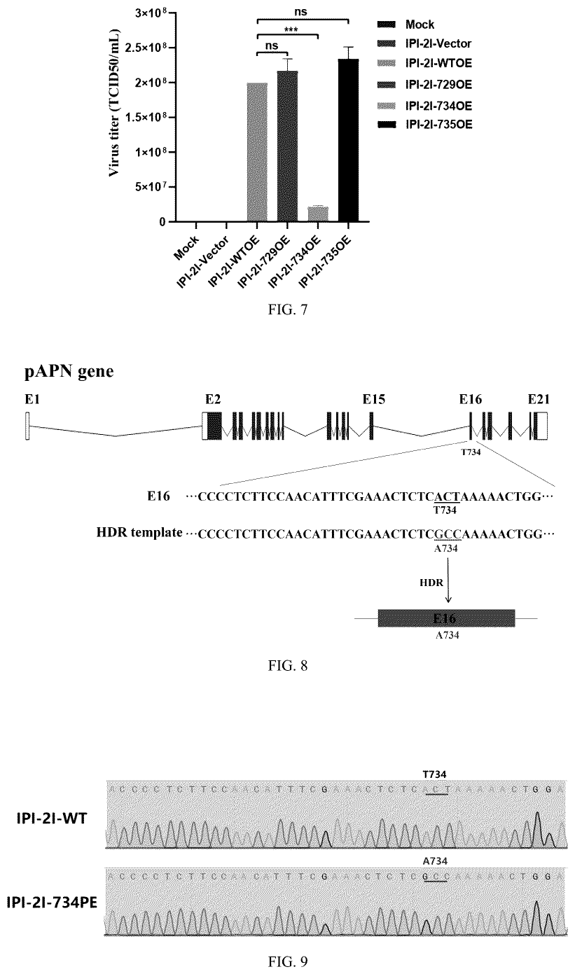

The TCID50 detection results were shown in FIG. 7, indicating that the TGEV virus titer was higher in IPI-2I-WTOE cells after virus inoculation; there was no significant change in virus titer in IPI-2I-729OE and IPI-2I-735OE cells, while the virus titer in IPI-2I-734OE cells was significantly reduced (***P<0.001) compared with IPI-2I-WT cells.

In summary, the results showed that both overexpressed porcine ileal epithelial cells with precise modification of amino acids at positions 729 and 735 in pAPN gene could not effectively resist TGEV infection, indicating that precise modification at any site in pAPN gene was not sufficient to resist TGEV infection; but overexpressed porcine ileal epithelial cells with precise modification of amino acids at position 734 in pAPN gene could effectively resist TGEV infection, indicating that the position 734 in pAPN gene was a key site for TGEV infection, and the precise modification of the amino acid at position 734 in pAPN gene could effectively resist TGEV infection.

### Example 2 Construction of an Expression Vector for a System of Site-Directed Modification at Position 734 in pAPN Gene

### I. Design of sgRNA Sequence and Construction of a Vector

1. The targeting site that was close to the amino acid site encoding T734 and had a higher score was selected with porcine pAPN gene as the sequence of interest using the sgRNA analysis tool CRISPOR (crispor.tefor.net), as shown below:

Sequence encoding pAPN-sgRNA-1:CTAGAAATACCTCAGGAAGC (SEQ ID NO: 2);

Sequence encoding pAPN-sgRNA-2:CGAGCGCCCAGAAAATCTGA (SEQ ID NO: 3).

sgRNA sequence synthesis:

Synthesis of complementary paired oligonucleotide sequences for pAPN-sgRNA-1 and pAPN-sgRNA-2 sequences:

pAPN-sgRNA-1-F:
(SEQ ID NO: 9)
caccgCTAGAAATACCTCAGGAAGC;
pAPN-sgRNA-1-R:
(SEQ ID NO: 10)
aaacGCTTCCTGAGGTATTTCTAGc;
pAPN-sgRNA-2-F:
(SEQ ID NO: 11)
caccgCGAGCGCCCAGAAAATCTGA;
pAPN-sgRNA-2-R:
(SEQ ID NO: 12)
aaacTCAGATTTTCTGGGCGCTCGc.

2. Construct the first and second vectors, named pX458-pAPN-sgRNA-1 and pX458-pAPN-sgRNA-2, respectively
(1) The complementary oligonucleotides corresponding to pAPN-sgRNA-1 and pAPN-sgRNA-2 in step 1 were treated at 98° C. for 10 min, respectively, and then naturally cooled to the room temperature for annealing.
(2) The pX458 backbone vector containing Cas9 sequence was cleaved with the restrictive endonuclease Bbs I at 37° C. for 2 h, followed by recovery of the linearized fragments by gel cutting.
(3) The above annealed double-stranded fragments were linked with the backbone of the linearized vector at 16° C. for 1 h, cooled in ice bath for 30 min, followed by heat shock for 45 s, and then transformed into Top10 or DH5α competent cells, which were coated and grown on LB plates containing ampicillin, and single colonies were picked on the next day for culturing and sequencing. Sequencing primers were as follows: U6-FWD: GAGGGCCTATTTCCCATGATT (SEQ ID NO: 13).
(4) The first (pX458-pAPN-sgRNA-1 plasmid) and second (pX458-pAPN-sgRNA-2 plasmid) vectors were extracted and cultured after sequences being correct, and frozen at −20° C. for subsequent cell transfection. The plasmid extraction was carried out using the EndoFree Plasmid Maxi Kit (CoWin Biotech, CW2104M).

### II. Design of Donor DNA Sequence and Construction of a Vector

### 1. Design of Donor DNA Sequence

Based on the sgRNA sequence, a donor DNA named pAPN-dsODN-734 with its specific sequence set forth in SEQ ID NO: 4 was designed for precise modification of the amino acid at position 734 in pAPN gene. The dsODN sequence shown was used as a double-stranded donor sequence, and when the double-stranded donor sequence replaced the wild-type sequence, T734 was successfully replaced with A734. A pattern diagram of precise mutation of the amino acid at position 734 in pig pAPN gene was shown as FIG. 8.
2. The pAPN-dsODN-734 obtained in part 1 of step II was linked into the PUC57 backbone vector (Genscript Biotech Corporation, SD1176) for sequencing validation, and the correctly sequenced recombinant plasmid was then amplified for subsequent cell transfection. The plasmid extraction was carried out using the EndoFree Plasmid Maxi Kit (CoWin Biotech, CW2104M).

### Example 3 Establishment and Functional Verification of the Monoclone from Porcine Ileal Epithelial Cells with Precise Modification of the Amino Acid at Position 734 in pAPN Gene

I. Establishment of the monoclone from porcine ileal epithelial cells with precise modification of the amino acid at position 734 in pAPN gene

Wild-type porcine ileal epithelial cells (IPI-2I-WT) were recovered into a 10 cm dish two days in advance, and the cell transfection could be performed until to 70-80% confluence of cells. 5 μg pX458-pAPN-sgRNA-1 plasmids, 5 μg pX458-pAPN-sgRNA-2 plasmids and 5 μg pAPN-dsODN plasmids were co-transfected into IPI-2I-WT cells with steps following strictly to the instructions of the Basic Primary Nucleofector Kit (Lonza, VPI-1002).

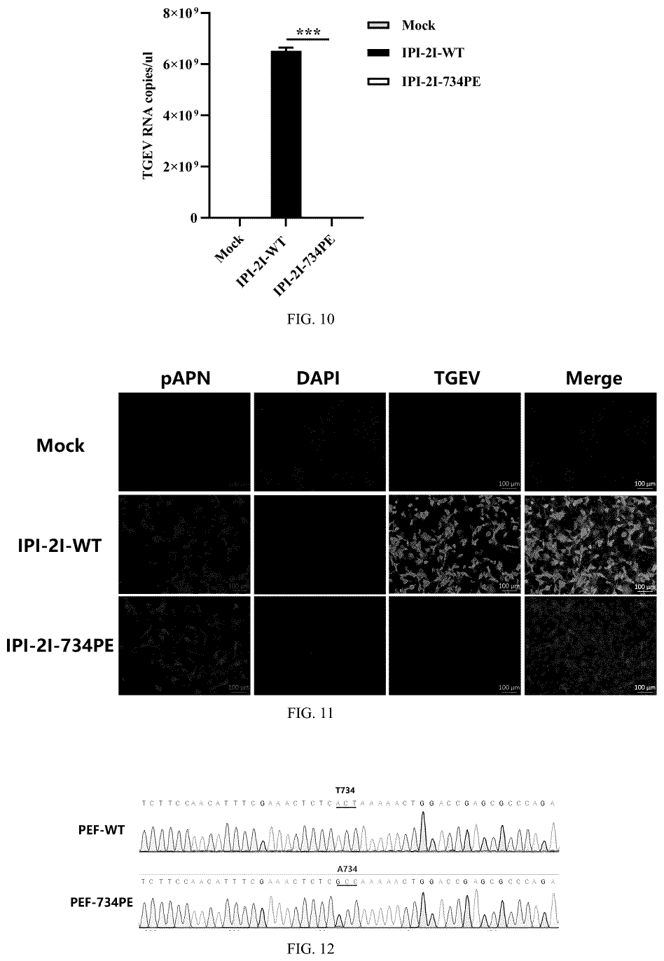

Cells were collected at 48 h after electrotransfection, followed by sorting into 96 well plates using a flow cytometric sorter for culture with the culture medium refreshed every 3 days. After cultivation of the sorted cells for about 10 days, the monoclonal cells were passaged to 48 well plates, and parts of cells were taken for genome extraction and genotype identification until full of cells in 48 well plates. The results showed that IPI-2I (IPI-2I-734PE) cells with precise modification of the amino acid at position 734 in pAPN gene were obtained (FIG. 9), which could be used as donor cells for subsequent TGEV infection experiments.
II. Functional verification of the monoclone from porcine ileal epithelial cells with precise modification of the amino acid at position 734 in pAPN gene

IPI-2I-734PE cells obtained above were tested for TGEV infection. IPI-2I-WT and IPI-2I-734PE cells were inoculated with TGEV virus strains (MOI=1), respectively, and IPI-2I-WT cells without virus inoculation were used as the Mock group.

Cells were collected at 12 h after infection, followed by extraction of RNA for detection of the copy number of TGEV virus in cells. The results of qPCR were shown in FIG. 10, indicating that the TGEV genome RNA replicated extensively on IPI-2I-WT cells; the copy number of TGEV genomic RNA in IPI-2I-734PE cells was significantly reduced (***P<0.001), compared with IPI-2I-WT cells.

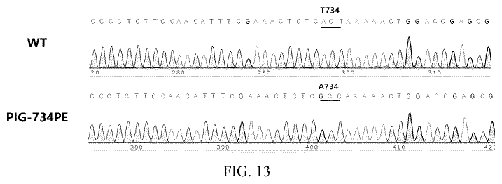

Cells were collected at 12 h after infection, followed by indirect immunofluorescence assay with the results shown as FIG. 11. The results indicated that IPI-2I-WT cells were infected with a large number of TGEVs after virus inoculation; while there was almost no TGEV infection in IPI-2I-734PE cells compared with IPI-2I-WT cells.

The above results showed that the monoclonal porcine ileal epithelial cells with precise modification of amino acids at position 734 in pAPN gene could effectively resist TGEV infection, indicating that the position 734 in pAPN gene was a key site for TGEV infection, and the precise modification of the amino acid at position 734 in pAPN gene could effectively resist TGEV infection.

### Example 4 Establishment of the Monoclone From Porcine Fibroblasts with Precise Modification of the Amino Acid at Position 734 in pAPN Gene

### I. Preparation of Porcine Fetal Fibroblasts

The head, tail, limbs, viscera, and bones from a pig embryo at 35 days old were removed, and the blood was cleaned. The fetus was continuously cut using an elbow ophthalmic scissor for 30 min to ensure sufficient fragmentation, and then the fetal tissue fragments were pipetted into a 15 mL centrifuge tube using the head-cutted blue tip, into which 5 mL of complete culture medium were added, followed by natural settlement for a few minutes to remove the supernatant, and a few drops of fetal bovine serum were added to the lower tissue block, which was sucked out using a 15 cm curved glass Pasteur tube bent at 1 cm from the tip, and placed into two T75 culture bottles with the bottom facing upwards, followed by addition of 15 mL of complete culture medium to the opposite side. The culture bottles were carefully turned over after 6-8 h, so that the tissue block was immersed in the culture medium with refreshment every two days to obtain cells, which were frozen and stored for future use until filling the T75 culture bottles. In this process, pigs were fed in the base pig farm of the Beijing Institute of Animal Science of CAAS.

### II. Cell Transfection

The primary porcine fetal fibroblasts were recovered into a 10 cm plate at the day before transfection, and the cell transfection could be performed until to about 80% confluence of cells. 5 μg pX458-pAPN-sgRNA-1 plasmids, 5 μg pX458-pAPN-sgRNA-2 plasmids and 5 μg Donor-734 plasmids were co-transfected into porcine fetal fibroblasts with steps following strictly to the instructions of the Basic Primary Fibroblasts Nucleofector Kit (Lonza), and then the electrotransfected cells were transferred to a 6-well plate for culture.

### III. Flow Cytometric Sorting and Passage of Monoclonal Cells

Cells were digested and collected into a tube for flow cytometry at 48 h after electroporation. Individual GFP positive cells were sorted using a flow cytometric sorter and cultured in a 96 well plate with refreshment of the culture medium every 3 days. Cells were passaged to a 48 well plate for culture until filling the 96 well plate, and then a portion of cells were taken for genome extraction and genotype identification until filling the 48 well plate.

### IV. Identification of Monoclonal Cells

Identification of picked monoclonal cells: the extracted DNA genome was amplified with the upstream and downstream primers set forth in SEQ ID NO: 14 (pAPN-TY-F2 sequence:CAAGGATTTGGAGGAGAA) and SEQ ID NO: 15 (pAPN-TY-R2 sequence:GCTGAGCGGAGTTTGTCG) to obtain a 1443 bp fragment, using the extracted cell genome as a template. The amplification condition was as follows: 94° C. for 5 min; 94° C. for 30 s, 62.6° C. for 30 s, 68° C. for 1 min 40 s, 34 cycles; 72° C. for 5 min. PCR products were sequenced by Beijing Tianyi Huiyuan Company. Based on the results of sequencing, porcine fibroblasts with precise modification of the amino acid at position 734 in pAPN gene were screened as donor cells for nuclear transplantation.

The results of sequencing showed that multiple strains of porcine fibroblasts (PEF-734PE) with precise modification of the amino acid at position 734 in pAPN gene were successfully obtained in this example, and some results of sequencing for the positive cells were shown as FIG. 12.

### Example 5 Preparation of a Genetically Edited Pig With Precise Modification of the Amino Acid at Position 734 in pAPN Gene by Somatic Cell Nuclear Transplantation Technology

The positive cells obtained from homozygous gene editing in Example 4 were used as donor cells for nuclear transfer, and the enucleated porcine oocytes matured in vitro for 40 h were used as recipient cells for nuclear transfer. The donor cells for nuclear transfer were transferred into the oocytes, which were electrically fused and activated to construct recombinant cloned embryos. The well-developed cloned recombinant embryos were selected and surgically transplanted into the uterus of naturally estrous multiparous white sows for pregnancy. In this process, steps of surgical embryo transfer were as follows: the recipient sow was anesthetized by intravenous injection of Zoletil with a dosage of 5 mg/kg body weight. After anesthesia, the recipient sows were moved to an operating rack for supine fixation, followed by respiratory anesthesia (with a concentration of 3% to 4% isoflurane). An about 10 cm long of surgical incision was made at the midline of the abdomen of the recipient sow to expose ovaries, fallopian tubes, and uterus. An embryo transfer glass tube was used to enter about 5 cm along the fimbria of the fallopian tubes, and the well-developed cloned recombinant embryos were transferred to the junction between the ampulla and isthmus of the fallopian tubes. Embryos were regularly observed by the technicians after transplantation, and the pregnancy statuses of the recipient sows were examined by B-type ultrasound.

Ear tissues were cut from piglets after birth, followed by extraction of genomic DNA, which was amplified by PCR using the above nucleotide sequences of SEQ ID NO: 14 and SEQ ID NO: 15, and the products from PCR amplification were sequenced for genotype detection.

The results of sequencing showed that the genetically edited pig (PIG-734PE) with precise modification of the amino acid at position 734 in pAPN gene was successfully obtained in this example, and some results of sequencing for the genetically edited pig were shown as FIG. 13.

Finally, it should be noted that the above examples are only used to illustrate the technical solutions of the present invention and not to limit it. Although the present invention has been described in detail with reference to the above examples, it should be understood by persons of ordinary skill in the art that the technical solutions recorded in the above examples may be modified or equivalently replaced some or all of the technical features. However, these modifications or replacements should not cause the essence of the corresponding technical solutions to deviate from the scope of the technical solutions of various examples in the present invention.

1-10. (canceled)

11. A pAPN mutant having an alanine at position 734, wherein the amino acid sequence of the pAPN mutant is forth in SEQ ID NO: 1.

12. (canceled)

13. A substance selected from the followings:
(1) a polynucleotide encoding the pAPN mutant of claim 11, wherein the polynucleotide encoding the mutation at position 734 in pAPN has a sequence of GCC;
(2) a composition for site-directed modification of pAPN gene, wherein the composition for site-directed modification of pAPN gene comprises a first sgRNA, a second sgRNA and a donor DNA; and the first and second sgRNAs each target two respective target sites in pAPN gene;
and the donor DNA mutating T734 to A734 in pAPN contains a site-directed modified fragment of an amino acid at position 734 in pAPN gene, which is located between the two respective target sites,
the nucleotide sequence encoding a gene at position 734 in pAPN gene has a sequence of GCC, which is achieved by the donor DNA; and
(3) a cell with site-directed modification of pAPN gene, wherein the pAPN mutated cell expresses the pAPN mutant.

14. (canceled)

15. The substance according to claim 13,
wherein the nucleotide sequence of the donor DNA is set forth in SEQ ID NO: 4;
preferably, the nucleotide sequence encoding the first sgRNA is set forth in SEQ ID NO: 2;
preferably, the nucleotide sequence encoding the second sgRNA is set forth in SEQ ID NO: 3.

16. The substance according to claim 15, wherein the composition comprises a first vector comprising an expression cassette for expressing the first sgRNA and a second vector comprising an expression cassette for expressing the second sgRNA;
preferably, the first vector further comprises an expression cassette for a genetically edited protein;
preferably, the second vector further comprises an expression cassette for a genetically edited protein;
preferably, the genetically edited proteins expressed by the first and second vectors independently comprise Cas9, Cas9n, Cpf1, or C2c2, respectively, and further independently preferably Cas9;
preferably, the backbones of the first and second vectors are independently derived from pX330, pX260, pX334, pX335, pX458, pX459, pX461, pX462, pX551, or pX552, respectively; and further independently preferably pX458.

17. A method selected from the followings:
(a) a method for constructing a cell line with site-directed modification of pAPN gene;
(b) a preparation method of a product for prevention of transmissible gastroenteritis in pigs;
(c) a method for constructing a pig model with transmissible gastroenteritis resistance;
(d) a preparation method of a cell with site-directed modification of pAPN gene, wherein the preparation method of a cell with site-directed modification of pAPN gene comprises that introducing a composition of a pAPN mutant having an alanine at position 734 into a cell of interest to obtain the cell with site-directed modification of pAPN gene; and
(e) a preparation method of a genetically edited pig, wherein the preparation method of a genetically edited pig comprises that transplanting the cell of the pAPN mutant into an enucleated oocyte to obtain a recombinant cloned embryo, which is transplanted into a maternal body for pregnancy to obtain a genetically edited pig with modification of an amino acid at position 734 in pAPN gene.

18. The method according to claim 17, wherein
the cell of interest is a porcine fibroblast or a porcine ileal epithelial cell;
preferably, a method for introducing comprises electroporation or liposome transfection;
further preferably electroporation;
preferably, the cell with site-directed modification of pAPN gene is obtained by screening and identification after the introduction operation;
preferably, the screening comprises screening a monoclonal cell by flow cytometric sorting;
preferably, the identification comprises sequencing or PCR identification;
preferably, PCR identification is performed using primers set forth in SEQ ID NOs: 14-15.

19. The substance according to claim 13, wherein the pAPN mutated cell contains a polynucleotide encoding the pAPN mutant; alternatively, it is prepared by a preparation method of a cell with site-directed modification of the pAPN gene.

20. The method according to claim 17, wherein the preparation method of a genetically edited pig can be replaced by microinjecting the composition of the pAPN mutant into a zygotic embryo in a pig by microinjection to obtain a pAPN gene-modified embryo, which is transplanted into a maternal body for pregnancy to obtain a genetically edited pig with modification of an amino acid at position 734 in pAPN gene;
preferably, a step of identification after birth is further comprised for the genetically edited pig;
preferably, the identification comprises sequencing or PCR identification;
preferably, PCR identification is performed using primers set forth in SEQ ID NOs: 14-15.

21. The method according to claim 17, wherein the composition for site-directed modification of pAPN gene of the pAPN mutant is used for any one of (a) to (e).


SEQUENCE LISTING (15 sequences)

SEQ ID NO: 1 - protein, 963 aa, Sus scrofa
MAKGFYISKALGILGILLGVAAVATIIALSVVYAQEKNKNAEHVPQAPTSPTITTTAAITLDQSKPWNRYRLPTTLLPDSYNVTLRPYLTPNADGLYIFKGKSIVRFICQEPTDVIIIHSKKLNYTTQGHMVVLRGVGDSQVPEIDRTELVELTEYLVVHLKGSLQPGHMYEMESEFQGELADDLAGFYRSEYMEGNVKKVLATTQMQSTDARKSFPCFDEPAMKATFNITLIHPNNLTALSNMPPKGSSTPLAEDPNWSVTEFETTPVMSTYLLAYIVSEFQSVNETAQNGVLIRIWARPNAIAEGHGMYALNVTGPILNFFANHYNTPYPLPKSDQIALPDFNAGAMENWGLVTYRENALLFDPQSSSISNKERVVTVIAHELAHQWFGNLVTLAWWNDLWLNEGFASYVEYLGADHAEPTWNLKDLIVPGDVYRVMAVDALASSHPLTTPAEEVNTPAQISEMFDSISYSKGASVIRMLSNFLTEDLFKEGLASYLHAFAYQNTTYLDLWEHLQKAVDAQTSIRLPDTVRAIMDRWTLQMGFPVITVDTKTGNISQKHFLLDSESNVTRSSAFDYLWIVPISSIKNGVMQDHYWLRDVSQAQNDLFKTASDDWVLLNINVTGYFQVNYDEDNWRMIQHQLQTNLSVIPVINRAQVIYDSFNLATAHMVPVTLALDNTLFLNGEKEYMPWQAALSSLSYFSLMFDRSEVYGPMKKYLRKQVEPLFQHFETLAKNWTERPENLMDQYSEINAISTACSNGLPQCENLAKTLFDQWMSDPENNPIHPNLRSTIYCNAIAQGGQDQWDFAWGQLQQAQLVNEADKLRSALACSNEVWLLNRYLGYTLNPDLIRKQDATSTINSIASNVIGQPLAWDFVQSNWKKLFQDYGGGSFSFSNLIQGVTRRFSSEFELQQLEQFKKNNMDVGFGSGTRALEQALEKTKANIKWVKENKEVVLNWFIEHS

SEQ ID NO: 2 - RNA, 20 nt, synthetic construct
  source 1..20: mol_type = other RNA
ctagaaatacctcaggaagc

SEQ ID NO: 3 - RNA, 20 nt, synthetic construct
  source 1..20: mol_type = other RNA
cgagcgcccagaaaatctga

SEQ ID NO: 4 - DNA, 1000 bp, synthetic construct
  source 1..1000: mol_type = other DNA
cctttgagcacagtctggccttgtgcgaggcctttagcctctggcctcttgctcctgtagccattagctcttgctacatctgcccacccacatcagaggctccatgggtctccagatgactcaggcatgagtctcttctttgaagctatttttagggctgcatcctcggcatgtggaggttcccaagctaggggttgaatcggagctgtagccgccagcctacaccacagccacagcaacacgggatccgagccacatctgcgacctacaccacagctcacagcaatgccagatccttaacccactgagtggggccagggttgaacccatgtcctcatgtttcccagtcagattcgtttctgctgtgccatgacgggaactctggaacttcctctttgaagctctttatgttttgttcttgttttttgtttttgtttttctagaaatacctcaggaagcaagtcgaacccctcttccaacatttcgaaactctcgccaaaaactggaccgagcgcccagaaaacttaatggaccagtgagtatgagctcgcttggtctggagatcatgggtggtgcaggtagcctgacctgggggcccatagcaagtccagcagcatcctctctggagctcccaactcctggccggaccagggccacagtcagggagagcgacccctcccaaccccactcccggccccaggagtagggactctgctctgaggctctgtgtggcctatgaaccatctggcctctttgggcaaaggaccaaactgaacctctgagggtccctcacccgcatggtgaggttctaggtgttaaagctggggctggagcctgtgccagccctccccaggctgcccaagggcaagaagcaaagaagggaacccaaaggtggctggtgggctatacctgcagagtgcgggtctgcctccctgttgggagttgtgtgtcagcaggggagtcttggtcagcgtcaggtccaggcgtgctgacagagtgt

SEQ ID NO: 5 - DNA, 2892 bp, Sus scrofa
  source 1..2892: mol_type = other DNA
atggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcatctgccaggagcccaccgatgtcatcatcatccatagcaagaagctcaactacaccacccaggggcacatggtggtcctgcggggcgtgggggactcccaggtcccagagatcgacaggactgagctggtagagctcactgagtacctggtggtccacctcaagggctcgctgcagcccggccacatgtacgagatggagagtgaattccagggggaacttgccgacgacctggcaggcttctaccgcagcgagtacatggagggcaacgtcaaaaaggtgctggccacgacacagatgcagtctacagatgcccggaaatccttcccatgctttgacgagccagccatgaaggccacgttcaacatcactctcatccaccctaacaacctcacggccctgtccaatatgccgcccaaaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctgatccggatctgggctcggcctaatgcaattgcagagggccatggcatgtatgccctgaatgtgacaggtcccatcctaaacttctttgccaatcattataatacaccctacccactccccaaatccgaccagattgccttgcccgacttcaatgccggtgccatggagaactgggggctggtgacctaccgggagaacgcgctgctgtttgacccacagtcctcctccatcagcaacaaagagcgagttgtcactgtgattgctcacgagctggcccaccagtggtttggcaacctggtgaccctggcctggtggaatgacctgtggctgaatgagggctttgcctcctatgtggagtacctgggtgctgaccacgcagagcccacctggaatctgaaagacctcatcgtgccaggcgacgtgtaccgagtgatggctgtggatgctctggcttcctcccacccgctgaccacccctgctgaggaggtcaacacacctgcccagatcagcgagatgtttgactccatctcctacagcaagggagcctcggttatcaggatgctctccaacttcctgactgaggacctgttcaaggagggcctggcgtcctacttgcatgcctttgcctatcagaacaccacctacctggacctgtgggagcacctgcagaaggctgtggatgctcagacgtccatcaggctgccagacactgtgagagccatcatggatcgatggaccctgcagatgggcttccccgtcatcaccgtggacaccaagacaggaaacatctcacagaagcacttcctcctcgactccgaatccaacgtcacccgctcctcagcgttcgactacctctggattgttcccatctcatctattaaaaatggtgtgatgcaggatcactactggctgcgggatgtttcccaagcccagaatgatttgttcaaaaccgcatcggacgattgggtcttgctgaacatcaacgtgacaggctatttccaggtgaactacgacgaggacaactggaggatgattcagcatcagctgcagacaaacctgtcggtcatccctgtcatcaatcgggctcaggtcatctacgacagcttcaacctggccactgcccacatggtccctgtcaccctggctctggacaacaccctcttcctgaacggagagaaagagtacatgccctggcaggccgccctgagcagcctgagctacttcagcctcatgttcgaccgctccgaggtctatggccccatgaagaaatacctcaggaagcaggtcgaacccctcttccaacatttcgaaactctcactaaaaactggaccgagcgcccagaaaatctgatggaccagtacagtgagattaatgccatcagcactgcctgctccaatggattgcctcaatgtgagaatctggccaagacccttttcgaccagtggatgagcgacccagaaaataacccgatccaccccaacctgcggtccaccatctactgcaatgccatagcccagggcggccaggaccagtgggactttgcctgggggcagttacaacaagcccagctggtaaatgaggccgacaaactccgctcagcgctggcctgcagcaacgaggtctggctcctgaacaggtacctgggttacaccctgaacccggacctcattcggaagcaagacgccacctccactattaacagcattgccagcaatgtcatcgggcagcctctggcctgggattttgtccagagcaactggaagaagctctttcaggactatggcggtggttccttctccttctccaacctcatccagggtgtgacccgaagattctcctctgagtttgagctgcagcagctggagcagttcaagaagaacaacatggatgtgggcttcggctccggcacccgggctctggagcaagccctggagaagaccaaggccaacatcaagtgggtgaaggagaacaaggaggtggtgttgaattggttcatagagcacagctaa

SEQ ID NO: 6 - DNA, 2892 bp, synthetic construct
  source 1..2892: mol_type = other DNA
atggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcatctgccaggagcccaccgatgtcatcatcatccatagcaagaagctcaactacaccacccaggggcacatggtggtcctgcggggcgtgggggactcccaggtcccagagatcgacaggactgagctggtagagctcactgagtacctggtggtccacctcaagggctcgctgcagcccggccacatgtacgagatggagagtgaattccagggggaacttgccgacgacctggcaggcttctaccgcagcgagtacatggagggcaacgtcaaaaaggtgctggccacgacacagatgcagtctacagatgcccggaaatccttcccatgctttgacgagccagccatgaaggccacgttcaacatcactctcatccaccctaacaacctcacggccctgtccaatatgccgcccaaaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctgatccggatctgggctcggcctaatgcaattgcagagggccatggcatgtatgccctgaatgtgacaggtcccatcctaaacttctttgccaatcattataatacaccctacccactccccaaatccgaccagattgccttgcccgacttcaatgccggtgccatggagaactgggggctggtgacctaccgggagaacgcgctgctgtttgacccacagtcctcctccatcagcaacaaagagcgagttgtcactgtgattgctcacgagctggcccaccagtggtttggcaacctggtgaccctggcctggtggaatgacctgtggctgaatgagggctttgcctcctatgtggagtacctgggtgctgaccacgcagagcccacctggaatctgaaagacctcatcgtgccaggcgacgtgtaccgagtgatggctgtggatgctctggcttcctcccacccgctgaccacccctgctgaggaggtcaacacacctgcccagatcagcgagatgtttgactccatctcctacagcaagggagcctcggttatcaggatgctctccaacttcctgactgaggacctgttcaaggagggcctggcgtcctacttgcatgcctttgcctatcagaacaccacctacctggacctgtgggagcacctgcagaaggctgtggatgctcagacgtccatcaggctgccagacactgtgagagccatcatggatcgatggaccctgcagatgggcttccccgtcatcaccgtggacaccaagacaggaaacatctcacagaagcacttcctcctcgactccgaatccaacgtcacccgctcctcagcgttcgactacctctggattgttcccatctcatctattaaaaatggtgtgatgcaggatcactactggctgcgggatgtttcccaagcccagaatgatttgttcaaaaccgcatcggacgattgggtcttgctgaacatcaacgtgacaggctatttccaggtgaactacgacgaggacaactggaggatgattcagcatcagctgcagacaaacctgtcggtcatccctgtcatcaatcgggctcaggtcatctacgacagcttcaacctggccactgcccacatggtccctgtcaccctggctctggacaacaccctcttcctgaacggagagaaagagtacatgccctggcaggccgccctgagcagcctgagctacttcagcctcatgttcgaccgctccgaggtctatggccccatgaagaaatacctcaggaagcaggtcgaacccctcttccaagccttcgaaactctcactaaaaactggaccgagcgcccagaaaatctgatggaccagtacagtgagattaatgccatcagcactgcctgctccaatggattgcctcaatgtgagaatctggccaagacccttttcgaccagtggatgagcgacccagaaaataacccgatccaccccaacctgcggtccaccatctactgcaatgccatagcccagggcggccaggaccagtgggactttgcctgggggcagttacaacaagcccagctggtaaatgaggccgacaaactccgctcagcgctggcctgcagcaacgaggtctggctcctgaacaggtacctgggttacaccctgaacccggacctcattcggaagcaagacgccacctccactattaacagcattgccagcaatgtcatcgggcagcctctggcctgggattttgtccagagcaactggaagaagctctttcaggactatggcggtggttccttctccttctccaacctcatccagggtgtgacccgaagattctcctctgagtttgagctgcagcagctggagcagttcaagaagaacaacatggatgtgggcttcggctccggcacccgggctctggagcaagccctggagaagaccaaggccaacatcaagtgggtgaaggagaacaaggaggtggtgttgaattggttcatagagcacagctaa

SEQ ID NO: 7 - DNA, 2892 bp, synthetic construct
  source 1..2892: mol_type = other DNA
atggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcatctgccaggagcccaccgatgtcatcatcatccatagcaagaagctcaactacaccacccaggggcacatggtggtcctgcggggcgtgggggactcccaggtcccagagatcgacaggactgagctggtagagctcactgagtacctggtggtccacctcaagggctcgctgcagcccggccacatgtacgagatggagagtgaattccagggggaacttgccgacgacctggcaggcttctaccgcagcgagtacatggagggcaacgtcaaaaaggtgctggccacgacacagatgcagtctacagatgcccggaaatccttcccatgctttgacgagccagccatgaaggccacgttcaacatcactctcatccaccctaacaacctcacggccctgtccaatatgccgcccaaaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctgatccggatctgggctcggcctaatgcaattgcagagggccatggcatgtatgccctgaatgtgacaggtcccatcctaaacttctttgccaatcattataatacaccctacccactccccaaatccgaccagattgccttgcccgacttcaatgccggtgccatggagaactgggggctggtgacctaccgggagaacgcgctgctgtttgacccacagtcctcctccatcagcaacaaagagcgagttgtcactgtgattgctcacgagctggcccaccagtggtttggcaacctggtgaccctggcctggtggaatgacctgtggctgaatgagggctttgcctcctatgtggagtacctgggtgctgaccacgcagagcccacctggaatctgaaagacctcatcgtgccaggcgacgtgtaccgagtgatggctgtggatgctctggcttcctcccacccgctgaccacccctgctgaggaggtcaacacacctgcccagatcagcgagatgtttgactccatctcctacagcaagggagcctcggttatcaggatgctctccaacttcctgactgaggacctgttcaaggagggcctggcgtcctacttgcatgcctttgcctatcagaacaccacctacctggacctgtgggagcacctgcagaaggctgtggatgctcagacgtccatcaggctgccagacactgtgagagccatcatggatcgatggaccctgcagatgggcttccccgtcatcaccgtggacaccaagacaggaaacatctcacagaagcacttcctcctcgactccgaatccaacgtcacccgctcctcagcgttcgactacctctggattgttcccatctcatctattaaaaatggtgtgatgcaggatcactactggctgcgggatgtttcccaagcccagaatgatttgttcaaaaccgcatcggacgattgggtcttgctgaacatcaacgtgacaggctatttccaggtgaactacgacgaggacaactggaggatgattcagcatcagctgcagacaaacctgtcggtcatccctgtcatcaatcgggctcaggtcatctacgacagcttcaacctggccactgcccacatggtccctgtcaccctggctctggacaacaccctcttcctgaacggagagaaagagtacatgccctggcaggccgccctgagcagcctgagctacttcagcctcatgttcgaccgctccgaggtctatggccccatgaagaaatacctcaggaagcaggtcgaacccctcttccaacatttcgaaactctcgccaaaaactggaccgagcgcccagaaaatctgatggaccagtacagtgagattaatgccatcagcactgcctgctccaatggattgcctcaatgtgagaatctggccaagacccttttcgaccagtggatgagcgacccagaaaataacccgatccaccccaacctgcggtccaccatctactgcaatgccatagcccagggcggccaggaccagtgggactttgcctgggggcagttacaacaagcccagctggtaaatgaggccgacaaactccgctcagcgctggcctgcagcaacgaggtctggctcctgaacaggtacctgggttacaccctgaacccggacctcattcggaagcaagacgccacctccactattaacagcattgccagcaatgtcatcgggcagcctctggcctgggattttgtccagagcaactggaagaagctctttcaggactatggcggtggttccttctccttctccaacctcatccagggtgtgacccgaagattctcctctgagtttgagctgcagcagctggagcagttcaagaagaacaacatggatgtgggcttcggctccggcacccgggctctggagcaagccctggagaagaccaaggccaacatcaagtgggtgaaggagaacaaggaggtggtgttgaattggttcatagagcacagctaa

SEQ ID NO: 8 - DNA, 2892 bp, synthetic construct
  source 1..2892: mol_type = other DNA
atggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcatctgccaggagcccaccgatgtcatcatcatccatagcaagaagctcaactacaccacccaggggcacatggtggtcctgcggggcgtgggggactcccaggtcccagagatcgacaggactgagctggtagagctcactgagtacctggtggtccacctcaagggctcgctgcagcccggccacatgtacgagatggagagtgaattccagggggaacttgccgacgacctggcaggcttctaccgcagcgagtacatggagggcaacgtcaaaaaggtgctggccacgacacagatgcagtctacagatgcccggaaatccttcccatgctttgacgagccagccatgaaggccacgttcaacatcactctcatccaccctaacaacctcacggccctgtccaatatgccgcccaaaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctgatccggatctgggctcggcctaatgcaattgcagagggccatggcatgtatgccctgaatgtgacaggtcccatcctaaacttctttgccaatcattataatacaccctacccactccccaaatccgaccagattgccttgcccgacttcaatgccggtgccatggagaactgggggctggtgacctaccgggagaacgcgctgctgtttgacccacagtcctcctccatcagcaacaaagagcgagttgtcactgtgattgctcacgagctggcccaccagtggtttggcaacctggtgaccctggcctggtggaatgacctgtggctgaatgagggctttgcctcctatgtggagtacctgggtgctgaccacgcagagcccacctggaatctgaaagacctcatcgtgccaggcgacgtgtaccgagtgatggctgtggatgctctggcttcctcccacccgctgaccacccctgctgaggaggtcaacacacctgcccagatcagcgagatgtttgactccatctcctacagcaagggagcctcggttatcaggatgctctccaacttcctgactgaggacctgttcaaggagggcctggcgtcctacttgcatgcctttgcctatcagaacaccacctacctggacctgtgggagcacctgcagaaggctgtggatgctcagacgtccatcaggctgccagacactgtgagagccatcatggatcgatggaccctgcagatgggcttccccgtcatcaccgtggacaccaagacaggaaacatctcacagaagcacttcctcctcgactccgaatccaacgtcacccgctcctcagcgttcgactacctctggattgttcccatctcatctattaaaaatggtgtgatgcaggatcactactggctgcgggatgtttcccaagcccagaatgatttgttcaaaaccgcatcggacgattgggtcttgctgaacatcaacgtgacaggctatttccaggtgaactacgacgaggacaactggaggatgattcagcatcagctgcagacaaacctgtcggtcatccctgtcatcaatcgggctcaggtcatctacgacagcttcaacctggccactgcccacatggtccctgtcaccctggctctggacaacaccctcttcctgaacggagagaaagagtacatgccctggcaggccgccctgagcagcctgagctacttcagcctcatgttcgaccgctccgaggtctatggccccatgaagaaatacctcaggaagcaggtcgaacccctcttccaacatttcgaaactctcactgccaactggaccgagcgcccagaaaatctgatggaccagtacagtgagattaatgccatcagcactgcctgctccaatggattgcctcaatgtgagaatctggccaagacccttttcgaccagtggatgagcgacccagaaaataacccgatccaccccaacctgcggtccaccatctactgcaatgccatagcccagggcggccaggaccagtgggactttgcctgggggcagttacaacaagcccagctggtaaatgaggccgacaaactccgctcagcgctggcctgcagcaacgaggtctggctcctgaacaggtacctgggttacaccctgaacccggacctcattcggaagcaagacgccacctccactattaacagcattgccagcaatgtcatcgggcagcctctggcctgggattttgtccagagcaactggaagaagctctttcaggactatggcggtggttccttctccttctccaacctcatccagggtgtgacccgaagattctcctctgagtttgagctgcagcagctggagcagttcaagaagaacaacatggatgtgggcttcggctccggcacccgggctctggagcaagccctggagaagaccaaggccaacatcaagtgggtgaaggagaacaaggaggtggtgttgaattggttcatagagcacagctaa

SEQ ID NO: 9 - DNA, 25 bp, synthetic construct
  source 1..25: mol_type = other DNA
caccgctagaaatacctcaggaagc

SEQ ID NO: 10 - DNA, 25 bp, synthetic construct
  source 1..25: mol_type = other DNA
aaacgcttcctgaggtatttctagc

SEQ ID NO: 11 - DNA, 25 bp, synthetic construct
  source 1..25: mol_type = other DNA
caccgcgagcgcccagaaaatctga

SEQ ID NO: 12 - DNA, 25 bp, synthetic construct
  source 1..25: mol_type = other DNA
aaactcagattttctgggcgctcgc

SEQ ID NO: 13 - DNA, 21 bp, synthetic construct
  source 1..21: mol_type = other DNA
gagggcctatttcccatgatt

SEQ ID NO: 14 - DNA, 20 bp, synthetic construct
  source 1..20: mol_type = other DNA
caaggatttgtggaggagaa

SEQ ID NO: 15 - DNA, 18 bp, synthetic construct
  source 1..18: mol_type = other DNA
gctgagcggagtttgtcg